(19) United States
(12) Patent Application Publication
(10) Pub. No.: US 2021/0277343 A1
(43) Pub. Date: Sep. 9, 2021

(54) RENEWABLE POWER TO RENEWABLE NATURAL GAS USING BIOLOGICAL METHANE PRODUCTION

(71) Applicant: Alliance for Sustainable Energy, LLC, Golden, CO (US)

(72) Inventors: Kevin William HARRISON, Erie, CO (US); Nancy Sue FARMER, Arvada, CO (US)

(21) Appl. No.: 17/261,473

(22) PCT Filed: Jul. 22, 2019
(86) PCT No.: PCT/US19/42861

§ 371 Date: Jan. 19, 2021

Related U.S. Application Data
(60) Provisional application No. 62/700,965, filed on Jul. 20, 2018.

(51) Int. Cl. C12M 1/107; C12P 5/02; C12M 1/00; C25B 1/04; C25B 15/08; C25B 9/73; C25B 9/05

(52) U.S. Cl. CPC C12M 21/04; C12P 5/023; C12M 43/06; C25B 9/05; C25B 15/083; C25B 9/73; C25B 1/04

(57) ABSTRACT

Systems, methods and devices for the production of renewable natural gasses are provided.

### CROSS-REFERENCE TO RELATED APPLICATIONS

This application claims priority under 35 U.S.C. § 371 to PCT application no. PCT/US2019/042861 with an international filing date of 22 Jul. 2019 which claims priority under 35 U.S.C. § 119 to U.S. Provisional Patent Application No. 62/700,965 filed on 20 Jul. 2018, the contents of both of which are hereby incorporated in their entirety.

### CONTRACTUAL ORIGIN

The United States Government has rights in this invention under Contract No. DE-AC36-08GO28308 between the United States Department of Energy and Alliance for Sustainable Energy, LLC, the Manager and Operator of the National Renewable Energy Laboratory.

This invention is CRADA work product under CRADA # CRD-14-567 between Alliance for Sustainable Energy, LLC on behalf of the National Renewable Energy Laboratory, and Southern California Gas Company.

### BACKGROUND

The cost of both wind- and solar-generated electricity have decreased significantly over the past decade with production costs approaching 2 cents per kilowatt-hour (kWh) level. While these energy prices have dropped, the capacity of renewables has taken off exponentially. As a consequence, certain states and regions of the world are curtailing—or having to shutdown these renewable electricity producers—to maintain a stable electrical grid. As these trends continue, hydrogen and other renewable fuels, like renewable methane—which also happens to recycle carbon dioxide—will become increasingly more economical due the abundance of low cost electricity and the need for long-duration energy storage.

Hydrogen gas is typically produced at pressures above ambient in today's commercially available water electrolyzer systems. That pressure of the hydrogen product from these systems can range from atmospheric to over 350 bar. However, most low temperature water electrolyzer systems being sold today range from 10-50 bar hydrogen output pressure. Pressurized hydrogen in the electrolyzer will be referred to as hydrogen, cathode or system pressure.

Deionized water (DIW) is typically fed to the electrolyzer stack on the anode side and direct current (DC) power splits water molecules into hydrogen and oxygen atoms. In the case of polymer electrolyte membrane (PEM) or proton exchange membrane (also, PEM), protons from the splitting are pulled from the anode to the cathode side of the electrolyzer cells under the influence of an applied voltage while also electro-osmotically dragging water molecules to the cathode. Electrolyzer systems then remove water accumulating on the cathode side of the electrolyzer cells. The two-phase, hydrogen/water mixture, flow on the cathode side then reaches a phase separator—separating the liquid water from the pressurized gas phase. Hydrogen gas, under pressure in the headspace of the phase separator, periodically pushes the water accumulating in the phase separator back into a larger water tank that is feeding the anode side of the electrolyzer cells. The vessel receiving the water and oxygen from the anode side of the cells is normally at a lower pressure than the hydrogen (cathode) side. Once the water from the hydrogen side meets the lower pressure atmosphere in the oxygen water side, hydrogen dissolved in the water comes out of solution and is swept out with the oxygen leaving the system. This phenomenon is central to this patent application. Normally, electrolyzer manufacturer's monitor the presence of hydrogen in oxygen as a safety measure.

Hydrogen gas, saturated with water vapor at an elevated temperature (40-80° C.), leaves the pressurized hydrogen/water phase separator and enters a pressure swing adsorption (PSA) drying system to remove the remaining water vapor from the hydrogen product gas. The PSA drying system normally consists of two parallel beds filled with desiccant to adsorb the water vapor contained in the hydrogen product gas stream. One of the two beds is active while the opposing bed is being regenerated using dry hydrogen, resulting in an efficiency loss for the electrolyzer system. The efficiency loss and use of dry hydrogen to regenerate the electrolyzer drying system is central to the innovation contained in this patent application.

SAE J2719 (Hydrogen Fuel Quality for Fuel Cell Vehicles) is a standard detailing the purity requirements for the hydrogen gas being used to fill fuel cell electric vehicles. Normally, the PSA system of the electrolyzer is sufficient to dry the hydrogen to less than 5 parts per million (ppm) by volume, which is the requirement of SAE J2719. In many electrolyzer systems, valuable hydrogen product gas is vented (i.e., wasted) as part of the PSA drying process to regenerate the parallel desiccant drying bed. To summarize the drying process of most commercially available electrolyzer systems, all of the product hydrogen gas enters the active drying bed. The hydrogen gas exiting the active bed is then used to dry (i.e., regenerate) the opposing bed. Because the hydrogen being used to dry the inactive bed now has picked up water vapor, that hydrogen gas does not meet the quality standard is vented from the system representing a loss of electrolyzer system efficiency between 3-10%.

### SUMMARY

In an aspect, disclosed is a method for the production of a gas comprising the use of an electrolyzer capable of producing pressurized hydrogen gas in an aqueous solution wherein the electrolyzer comprises a water/oxygen phase separator, a pump, an electrolyzer stack, and a back pressure regulator and wherein the electrolyzer does not comprise a hydrogen drying system or a hydrogen/water phase separator In an embodiment, the gas is a biogas. In another embodiment, the biogas is methane. In another embodiment, the pressurized hydrogen gas in an aqueous solution that is provided to a bioreactor comprising a biocatalyst In another embodiment, the biocatalyst catalyzes the production of the gas. In another embodiment, the biocatalyst is Methanothermobacter thermautotrophicus. In another embodiment, the aqueous solution is alkaline. In another embodiment, the aqueous solution comprises KOH or NaOH. In another embodiment, the pressurized hydrogen gas in an aqueous solution is used to control the pH of an aqueous solution in the bioreactor. In another embodiment, a carbon containing gas and the pressurized hydrogen gas in an aqueous solution are provided to the bioreactor. In another embodiment, the method further comprises the production of hydrogen gas. In another embodiment, the hydrogen gas and hydrogen dissolved in an aqueous solution is provided to the bioreactor directly from the electrolyzer stack.

In an aspect, disclosed is an electrolyzer capable of producing pressurized hydrogen gas in an aqueous solution wherein the electrolyzer comprises a water/oxygen phase separator, a pump, an electrolyzer stack, and a back pressure regulator and wherein the electrolyzer does not comprise a hydrogen drying system or a hydrogen/water phase separator.

In another aspect, disclosed is a system for the production of a gas comprising an electrolyzer capable of producing pressurized hydrogen gas in an aqueous solution wherein the electrolyzer comprises a water/oxygen phase separator, a pump, an electrolyzer stack, and a back pressure regulator and wherein the electrolyzer does not comprise a hydrogen drying system or a hydrogen/water phase separator and wherein the system further comprises a bioreactor that uses the pressurized hydrogen gas in an aqueous solution and a carbon containing gas and a biocatalyst in an aqueous solution to produce the gas. In an embodiment, the gas is methane. In an embodiment, the carbon containing gas is carbon dioxide. In an embodiment, the biocatalyst is Methanothermobacter thermautotrophicus. In an embodiment, the pressurized hydrogen gas in an aqueous solution is used to control the pH of an aqueous solution within the bioreactor. In an embodiment, the pressurized hydrogen gas in an aqueous solution is alkaline. In an embodiment, the aqueous solution comprises KOH or NaOH.

### BRIEF DESCRIPTION OF THE DRAWINGS

FIG. 1 depicts a schematic of an existing system of producing and drying hydrogen to a purity level required by fuel cells. As depicted in FIG. 1, Flows (1) is water (or electrolyte) entering into the stack from water pump; (2) water (or electrolyte) and oxygen from the stack return to the oxygen/water phase separator (3); oxygen and some hydrogen at near ambient pressure, due to the recycling of water containing dissolved hydrogen from the pressurized phase separator on the cathode side of the electrolyzer stack. The hydrogen comes out of solution when leaving the higher pressure hydrogen/water phase separator (C) and enters the lower pressure oxygen/water phase separator. (A) is a two-phase flow of hydrogen, water vapor and liquid water (stack cathode); (B) is hydrogen gas saturated with water vapor at pressure and a temperature typically in the range of 40-80° C.; (C) is water containing dissolved hydrogen which is returned (i.e., recycled) to the lower pressure oxygen/water phase separator; (D) is dried hydrogen product gas from the drying system; (E) is hydrogen containing water vapor from the drying bed being regenerated; (F) is hydrogen product gas exiting the electrolyzer to a downstream process, no longer limited to the set point of the electrolyzer system back pressure regulator. In an embodiment, the hydrogen gas is dried to less than 5 ppm of water vapor.

FIG. 2 depicts a schematic of an embodiment of the present invention. Flow (1) is water (or electrolyte) into the stack from water pump; flow (2) is water (or electrolyte) and oxygen from the stack return to the oxygen/water phase separator; flow (3) is oxygen and some hydrogen, due to the recycling of water containing dissolved hydrogen from the pressurized phase separator on the cathode side of the electrolyzer stack. The hydrogen comes out of solution when leaving the higher pressure hydrogen/water phase separator and then the flow enters the lower pressure oxygen/water phase separator. FIG. 2 depicts (A) two-phase flow of hydrogen, water vapor and liquid water (stack cathode); and (F) pressurized hydrogen gas, hydrogen gas dissolved in liquid water and water vapor (same as A) exiting the electrolyzer to feed downstream process requiring improvement in mass transfer of hydrogen.

FIG. 3 depicts an embodiment of an electrolyzer system configuration. In an embodiment, and as depicted in FIG. 3, the electrolyzer bed configuration includes a deionized water/oxygen phase separator, a deionized water pump, a heat exchanger, an electrolyzer stack, a hydrogen/water phase separator, a PSA hydrogen dryer system and a DC J-Box that brings in power from the AC/DC power supplies to the electrolyzer stack. As depicted in FIG. 3, the electrolyzer system can operate at from 20-70 bar, 4000 Adc at 250 Vdc, <5 ppmv H₂Ov; and produces about 5 kg H₂ hr using 250 kW PEM stack.

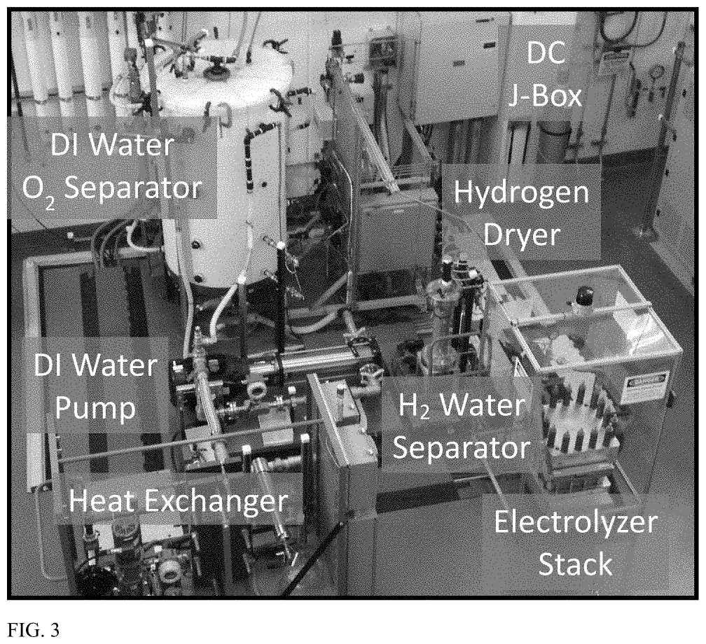

### DETAILED DESCRIPTION

Disclosed herein are systems, methods and devices for improving the production yields and rates for products produced from hydrogen taken before the drying steps (e.g. methane) and other products through increased hydrogen mass transfer in anaerobic gas fermentation systems. Disclosed herein are systems and methods utilizing hydrogen produced from an electrolyzer without the drying equipment. When coupled with a carbon dioxide (CO₂) source (i.e., biogas), and used under steady-state and variable input (CO₂ & H₂) gas flow can generate a product (i.e. pipeline quality natural gas (>95% CH₄) using a biocatalyst such as Methanothermobacter thermautotrophicus. In an embodiment, biomethanation uses inputs of H₂, CO₂ and nutrients (i.e., salts) and having outputs of CH₄, H₂O, and heat by using a biocatalyst such as Methanothermobacter thermautotrophicus.

### Definitions

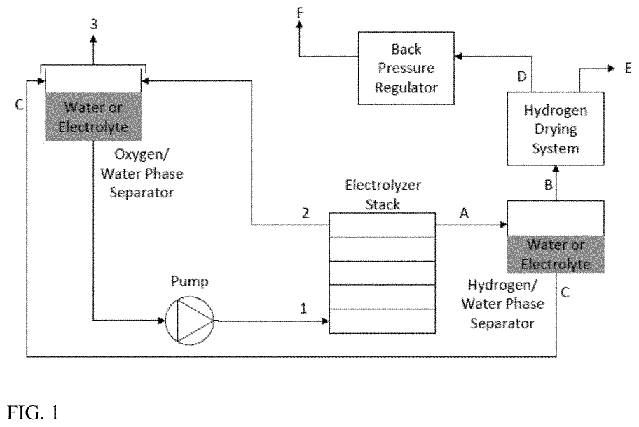

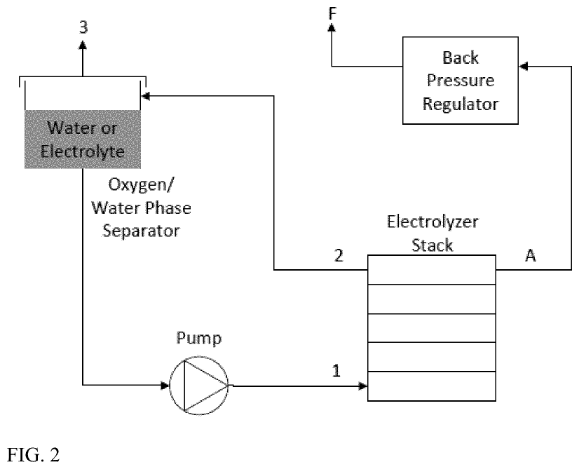

Electrolyzer stack—Electrochemical device made of a number of cells where water molecules are split to hydrogen at the cathode and oxygen at the anode. As depicted in FIGS. 1 and 2, this configuration shows a stack with water being fed to the anode at the bottom (1). Water and oxygen return to the oxygen/water phase separator from (2).

Oxygen/Water Phase Separator—Vessel, normally near atmospheric pressure, where water (or electrolyte) is supplied by the pump to the stack and where primarily oxygen is separated from the water feed. However, as depicted in FIGS. 1 and 2, when the water under pressure from flow C enters this lower pressure vessel hydrogen comes out of solution and exits via flow 3 from the vessel.

Back Pressure Regulator—Mechanical device maintaining hydrogen pressure back to the electrolyzer cathode, where hydrogen is created under pressure. Electrochemical compression of the hydrogen gas at the stack comes with a small voltage increase at the stack. This approach reduces the need for further compression of the hydrogen gas if you are feeding the downstream device at pressures at or lower than that of the electrolyzer. In other words, an electrolyzer stack operating at 20 bar could be closely coupled to a bioreactor vessel operating at 18 bar or lower, thus removing the need for mechanical compression of the hydrogen between the two devices.

Hydrogen/Water Phase Separator—Pressure vessel where liquid water that is pulled across from the anode-fed water supply is separated from the hydrogen gas. Water accumulates in the vessel until a level monitoring system initiates causing a valve to open which allows the hydrogen pressure to push the water back to the oxygen/water phase separator.

Hydrogen Drying System—Normally utilizes a desiccant material that adsorbs water vapor on to a material. Typically, two desiccant beds are operated in parallel where one is active and the other being regenerated. The active bed accepts all of the hydrogen gas flow from the electrolyzer stack which is saturated with water vapor based on the gas temperature. Some of the dry hydrogen from the active bed is ported to the bed being regenerated to sweep out the water vapor being released from the desiccant not at lower pressure. The active bed is under pressure from the back pressure regulator and the bed being regenerated is under near ambient pressure conditions.

Biocatalyst—In an embodiment, a biocatalyst is an organism that catalyzes a reaction of interest. A biocatalyst can be an enzyme or set of enzymes within an organism that catalyze reactions or a reaction of interest. In another embodiment, a biocatalyst can be any combination of an enzyme, polypeptide, polynucleotide or other biologically derived molecule. The enzyme, polynucleotide are biologically active.

### PEM Electrolyzer Systems

Proton exchange membrane or polymer electrolyte membrane (both abbreviated, PEM) electrolyzers typically operate at high differential pressures between the water/oxygen (anode) and the hydrogen (cathode) sides of the cells.

Hydrogen gas, from commercially available PEM-based electrolyzer systems, is typically delivered at pressures in the range of 10-50 bar, but systems have demonstrated higher pressures in the range of 50-350 bar. As a consequence, pressurized electrolyzer stacks operate at a higher voltage the ambient pressure stacks.

Electrochemical compression at the electrolyzer stack is expected to reach 700-900 bar to support direct refueling of fuel cell electric vehicles in the future.

PEM electrolyzer stacks consist of multiple cells and many commercially available electrolyzer systems contain many stacks to increase the hydrogen production from a single unit.

A single or multiple stack configuration becomes part of an electrolyzer system, which includes a balance of plant (BoP) of power supplies, hydrogen purification, main water loop, gas/liquid phase separators, safety and controls systems.

In PEM electrolyzer systems where water is fed into the anode, protons migrating across the solid membrane electro-osmotically drag water from the anode to the cathode.

Two phase flow of hydrogen gas and water from the electrolyzer stack requires clean-up prior to downstream compression or any other end-use application needing dry hydrogen product gas. However, this patent application challenges this approach by enabling the direct coupling of the electrolyzer stack to the bioreactor, eliminating the pressure vessel used as the hydrogen/water phase separator and the desiccant drying bed used to achieve low water vapor content in the product gas. In the elimination of these two sub-systems of the electrolyzer system capital cost is reduced and efficiency is increase for the electrolyzer system.

The first step towards hydrogen purification is accomplished via a gas/liquid phase separator. This patent application eliminates this requirement typically found in all of the commercially available electrolyzer systems.

Liquid water accumulates after the electrolyzer stack and is recycled by using the hydrogen pressure to push the water back towards the main deionized water loop. This action is a source of efficiency loss, due to the pressurized hydrogen dissolved in this water being moved back to the anode (oxygen) side of the electrolyzer cells. This patent application removes this requirement because the water containing the dissolved hydrogen can be immediately utilized by the organisms (biocatalysts) in the downstream bioreactor system. In other words, the organisms require gasses dissolved in water and the water coming from the pressurized cathode of an electrolyzer stack already contains hydrogen dissolved in the water. This patent application takes advantage of this fact and will result in the improved productivity of the organisms in the bioreactor and reduced mixing power load of the agitator on the bioreactor.

When water containing dissolved hydrogen from the pressurized cathode side of the electrolyzer stack is moved to a vessel at lower pressure (like the oxygen/water phase separator), hydrogen effervesces or is released from the water. The hydrogen that comes out of solution is vented from the system or exits the system along with the oxygen. This hydrogen loss reduces the efficiency of an electrolyzer system.

The BoP of commercially available electrolyzer systems normally includes a drying system that removes nearly all of the remaining water vapor from the product gas. Codes and standards, like SAE J2719, require water vapor content in the hydrogen gas to be less than 5 ppm by volume for fuel cell applications. Most electrolyzer manufacturers provide this level of product gas clean up.

Depending on the application, the water vapor in the resulting hydrogen product gas is reduced to less than 5 parts per million by volume (ppmv) to support fuel cell electric vehicle refueling. Biomethanation and other large-scale industrial end-use applications, however, do not require this level of clean up.

Starting at the output of the electrolyzer stack cathode, hydrogen dissolves in the water on the cathode side of the electrolyzer stack at elevated pressures.

### Alkaline Electrolyzer Systems

Alkaline electrolyzers that use a liquid electrolyte, like potassium hydroxide (KOH) and in other systems using sodium hydroxide (NaOH), operate with balanced pressure across the anode and cathode of the cells in the range of atmospheric to 50 bar.

Liquid alkaline electrolyzer systems have also attempted to achieve 400 bar balanced pressure operation, with limited success. Electrolyte on the cathode and anode are circulated to the stack separately. Pressurized product gases (i.e., oxygen and hydrogen) dissolve in the liquid electrolyte in each vessel on the anode and cathode sides.

### Biological Gas Fermentation Systems

Living organisms act as biocatalysts to convert input gases (e.g., hydrogen, carbon dioxide, carbon monoxide) to other products. Bioreactor systems can be designed at higher operating pressures to improve hydrogen mass transfer. The organisms require the hydrogen gas to be dissolved in the media (typically water) to utilize them in the reaction to other products. This technology integrates with pressurized bioreactor systems. However, higher pressures may be attainable and cost-effective for this and other biological upgrading systems—beyond the narrow application of production of methane in this biomethanation process.

Besides pressure, other approaches like agitation and water recirculation are employed aimed at improving hydrogen mass transfer to the organisms. Agitation inside the reactor breaks large bubbles down to smaller and smaller bubbles to help overcome the challenges with hydrogen solubility in the media (i.e., water). Counter-flow water recirculation works to keep the hydrogen and other feed gases suspended in the water longer. While gas bubbles are rising in the bioreactor, the water flow direction is in the downward direction to increase bubble residence time.

This technology can be used for other products, not just methane production. This technology can be applied to any gas fermentation system that utilizes wet hydrogen. In an embodiment, disclosed herein are new methods for integrating water electrolysis with a pressurized bioreactor in a manner that improves overall efficiency while reducing capital and operating costs. Systems and methods disclosed herein make integration of electric and gas grid operations technically and economically more viable.

Systems and methods disclosed herein are not only useful with regard to the generation of CH₄, but are also applicable to all gas fermentation processes requiring H₂ gas. In an embodiment, biomethanation is one gas fermentation process that benefits by using the systems and methods disclosed herein by reducing the capital cost of electrolyzer 2-10%; increasing the system efficiency of the electrolyzer by about 1-5% efficiency over commercial electrolyzer systems; and improving the H₂ mass transfer and therefore the conversion rate of the biocatalyst in the bioreactor.

The concepts being developed here can be applied to other processes. For example, nitrate is one of the most common groundwater contaminants impacting rural communities. Nutrient pollution has impacted many streams, rivers, lakes, bays and coastal waters for the past several decades, resulting in serious environmental and human health issues, and impacting the economy. Nitrate in groundwater originates primarily from fertilizers, septic systems, and manure storage or spreading operations. Water clean up companies use hydrogen derived from either natural gas via steam methane reforming or water electrolysis to remediate nitrate contaminated water. Water containing hydrogen is pumped through a biofilm reactor. One challenge these companies face is that hydrogen has very low solubility in water. This challenge could be overcome by passing the hydrogen directly from the electrolyzer stack into the aqueous media and into the biofilm. The proposed innovations would improve the efficiency of the water clean up process while also eliminating the cost of hydrogen gas compression equipment. Similarly, the electrolyte (potassium hydroxide) found in liquid alkaline electrolyzer systems contain dissolved hydrogen on the cathode sides of the cells. The two-phase solution from the alkaline electrolyzer stack could also help maintain pH of a downstream process.

As an additional benefit over existing electrolyzer system configurations is that operational safety is improved as the hydrogen dissolved in the water of the phase separator is no longer recycled to the oxygen/water (O₂H₂O) phase separator. There, the H₂ comes out of solution and is vented from the system along with the O₂.

In processes where the first step involves H₂ production under pressure greater than ambient in contact with liquid, in either a polymer electrolyte membrane (PEM) (i.e., water) or alkaline (i.e., liquid electrolyte) electrolyzer, H₂ gas will dissolve into the aqueous phase. In an embodiment, the two-phase flow of H₂ dissolved in the liquid and vapor phases improves the mass transfer of the hydrogen and results in higher productivity and efficiency of downstream processes.

The biomethanation process and other downstream H₂ uses are challenged with H₂ mass transfer rates because of hydrogen's inherently low solubility in water. In an embodiment, an improvement to processes like these would be to improve H₂ mass transfer so that the biocatalysts can metabolize the gas quicker and improve conversion efficiency. If the H₂ pressure from the stack cathode is slightly higher than the bioreactor pressure, H₂ gas and the H₂ dissolved in the liquid and water vapor will flow to the reactor without any further compression or cleanup. The H₂ dissolved in the liquid and water vapor coming from the stack will be more accessible to the biocatalysts for conversion.

CO₂+4H₂ plus Biocatalyst to CH₄+2H₂O+Heat Equation 1

Equation 1 depicts the stoichiometry of the reaction and how the biocatalyst use carbon from CO₂ to produce a synthetic fuel using renewable H₂.

In an embodiment, a significant portion of an electrolyzer balance of plant (BoP) needed for H₂ gas purification is eliminated by using methods and processed disclosed herein. The capital cost of the electrolyzer can be reduced by an estimated 2-10% with the elimination of the pressure swing adsorption H₂ drying system; the preventive maintenance and replacement desiccant that is no longer needed; the pressure vessel used as the gas/liquid phase separator on the cathode side; and the liquid level monitoring and valving between the pressurized vessel and the near-ambient pressure water/oxygen vessel.

By altering existing commercially available electrolyzer systems using the methods and systems disclosed herein, the efficiency of commercially available electrolyzer systems can be increased by an estimated 3-10%. Alterations to commercially available electrolyzer systems include with the following changes: eliminating the use of 3-5% of H₂ gas that is used to regenerate the desiccant needed to provide less than 5 ppmv H₂Ov for fueling applications. The H₂ dissolved in the water of the cathode phase separator can now be put to use in any downstream process challenged by H₂ mass transfer. Eliminating the recirculation of the water containing dissolved H₂, normally returned to O₂H₂O phase separator and vented with the O₂, also improves the safety of the electrolyzer operation.

In an embodiment, and as depicted in FIG. 2, in step two of the two-step process, bioreactor conversion rate is expected to increase by using the two-phase flow (i.e., H₂ gas and H₂ dissolved in water) directly from the electrolyzer stack. In addition, less agitation and water circulation power are required at the bioreactor to achieve the same biocatalyst conversion rate than dry H₂.

The H₂ drying losses of about 3-5% that exist in commercial electrolyzers are avoided by using systems and methods disclosed herein. This results in an overall electrolyzer system efficiency increase. Improvements in efficiency occur by bypassing both the H₂/H₂O phase separator as well as the H₂ desiccant drying system. This increases the electrolyzer system efficiency by using the H₂ dissolved in the water that normally accumulates and is recycled back to the O₂/H₂O phase separator. There, the dissolved H₂ comes out of solution when it hits the low pressure atmosphere of the O₂/H₂O phase separator and is vented along with the O₂ leaving the system. In an embodiment, optimization of the processes disclosed herein include balancing and controlling the additional water that the bioreactor receives from the electrolyzer.

In processes where the first step involves hydrogen production under pressure greater than ambient in contact with liquid in either a PEM (i.e., water) or alkaline (i.e., liquid electrolyte) electrolyzer, hydrogen gas will dissolve into solution.

In an embodiment of systems disclosed herein, a second, downstream process, will use the two-phase flow of hydrogen dissolved in the liquid water and water vapor to improve the mass transfer of the hydrogen resulting in higher productivity and/or efficiency of the downstream process.

A significant portion of the PEM electrolyzer system balance of plant (BoP) needed for gas purification is eliminated by using systems and methods disclosed herein. A downstream system from the electrolyzer is improved by the dissolved hydrogen gas in the two-phase flow from the stack, allowing for the removal of both the hydrogen gas/liquid water phase separator and an entire drying system that captures and recirculates the liquid water and removes the water vapor from the product gas.

In an embodiment, the capital cost of the electrolyzer is reduced by about 2-10% with the elimination of the pressurized hydrogen gas/liquid water phase separator, level monitoring and control valving and piping, thus significantly reducing the BoP associated with hydrogen gas clean up prior to delivery to a downstream system.

In an embodiment, the efficiency of the electrolyzer system is increased by about 2-10% by eliminating the loss of the hydrogen gas that becomes dissolved in the water contained in the gas/liquid phase separator typically located immediately after the electrolyzer stack in commercially available systems; and eliminating the hydrogen gas loss associated with regenerative (e.g., pressure swing adsorption) drying systems found in many commercially available electrolyzer systems.

By using systems and methods disclosed herein, the safety of a PEM electrolyzer system is improved by avoiding the mixing of water with dissolved hydrogen and the water feed/oxygen side of the stack where hydrogen comes out of solution and is either vented directly or mixes with oxygen and vented.

In an embodiment, the hydrogen output from the PEM stack cathode can be directly connected to a bioreactor to supply hydrogen to biocatalysts for conversion to fuels and chemicals without any further purification or water (liquid vapor) removal. If the hydrogen pressure from the stack cathode is higher than the bioreactor pressure, hydrogen gas and the hydrogen dissolved in the liquid and water vapor will flow to the reactor without any further compression.

In an embodiment, the hydrogen dissolved in the liquid and water vapor coming from the stack will be more readily accessible to the biocatalysts for conversion to a variety of products as long as pressure is maintained in the bioreactor.

By using systems and methods disclosed herein, less agitation and water circulation power will be required at the bioreactor to achieve the same organism productivity than the dry hydrogen without the water from the stack entering the bioreactor. Furthermore, bioreactor system efficiency will be increased using the two-phase flow (i.e., hydrogen gas and hydrogen dissolved water) directly from the electrolyzer stack.

By using systems and methods disclosed herein, the KOH and NaOH from liquid alkaline electrolyte electrolyzer systems will contain dissolved hydrogen on the cathode sides of the cells. The two phase solution from the alkaline electrolyzer stack can be used to help maintain pH of the system (an example being maintaining the pH setpoint in a bioreactor). In addition, the alkali containing dissolved hydrogen can supply nutrients to the biocatalysts.

In an embodiment, the systems and methods disclosed herein can be applied to any process (biological or chemical) that requires hydrogen to be entrained in liquid and under pressure.

In an embodiment, the hydrogen dissolved in the water from the electrolyzer is directly coupled with the downstream gas fermentation process where biocatalysts (i.e., organisms) take advantage of the dissolved gas to improve the productivity of whatever product they are making. For example, a biomethanation process may utilize methanogen archaea to convert carbon dioxide (CO₂) and hydrogen (H₂) into methane (CH₄).

The solubility of hydrogen in water is much lower than CO₂ and therefore one of the major challenges in anaerobic gas fermentation processes is the mass transfer of hydrogen to the organisms. Increasing the pressure of these processes improves the H₂ mass transfer by creating smaller bubbles and dissolved gasses for the organisms to metabolize. High pressure hydrogen gas, along with hydrogen dissolved in the water from the electrolyzer process, would improve the mass transfer of the process and increase the organism's productivity.

Hydrogen mass transfer, knocking big bubbles into very small bubbles so organisms can metabolize them, is very challenging in gas fermentation processes. Electrolyzer systems typically operate with the hydrogen side pressurized. Hydrogen dissolves in the water that is pulled from the anode to the cathode side. This water, with dissolved hydrogen, can directly enter a biological gas fermentation process where the biocatalysts will take advantage of the already dissolved hydrogen gas. This improves mass transfer in the pressurize reactor and would increase the productivity of the organisms. The biocatalysts, for example, may be metabolizing carbon dioxide and hydrogen to produce methane. The dissolved hydrogen in the water coming from the electrolyzer will improve the organism's productivity of methane production, in this case.

In an embodiment, disclosed is a method to closely- or directly-couple an electrolyzer system producing pressurized hydrogen gas to a downstream process that would be improved by the presence of hydrogen dissolved in water or other electrolyte.

In an embodiment, hydrogen dissolved in another solution, like aqueous KOH or NaOH in an alkaline electrolyzer, can be used by the downstream process for pH control. For example, pH control in a bioreactor. In addition, the potassium and sodium could utilized by systems requiring these ions as nutrients.

The method eliminates sub-systems of an electrolyzer system to reduce capital costs while improving system efficiency by eliminating hydrogen loss in the gas clean up systems composed of a pressurized hydrogen/water phase separator and desiccant or other drying technique.

As disclosed herein the approaches also improve electrolyzer safe operations by eliminating the hydrogen coming out of solution in the presence of oxygen on the anode side of the stack. This hydrogen, which is dissolved in water, is provided directly to the downstream process for use, instead of being vented with the oxygen byproduct.

The methods disclosed herein improve downstream processes like biomethanation by providing hydrogen dissolved in water or an electrolyte thus increasing gas mass transfer that becomes immediately accessible to biocatalysts for improved conversion rates.

The foregoing disclosure has been set forth merely to illustrate the invention and is not intended to be limiting. Since modifications of the disclosed embodiments incorporating the spirit and substance of the invention may occur to persons skilled in the art, the invention should be construed to include everything within the scope of the appended claims and equivalents thereof

Other objects, advantages, and novel features of the present invention will become apparent from the detailed description of the invention when considered in conjunction with the accompanying drawing and attached appendix.

What is claimed is:

1. A method for the production of a gas comprising the use of an electrolyzer capable of producing pressurized hydrogen gas in an aqueous solution wherein the electrolyzer comprises a water/oxygen phase separator, a pump, an electrolyzer stack, and a back pressure regulator and wherein the electrolyzer does not comprise a hydrogen drying system or a hydrogen/water phase separator.

2. The method of claim 1 wherein the gas is a biogas.

3. The method of claim 2 wherein the biogas is methane.

4. The method of claim 1 wherein the pressurized hydrogen gas in an aqueous solution is provided to a bioreactor comprising a biocatalyst.

5. The method of claim 4 wherein the biocatalyst catalyzes the production of the gas.

6. The method of claim 5 wherein the biocatalyst is Methanothermobacter thermautotrophicus.

7. The method of claim 4 wherein the aqueous solution is alkaline.

8. The method of claim 7 wherein the aqueous solution comprises KOH or NaOH.

9. The method of claim 4 wherein the pressurized hydrogen gas in an aqueous solution is used to control the pH of an aqueous solution in the bioreactor.

10. The method of claim 5 wherein a carbon containing gas and the pressurized hydrogen gas in an aqueous solution are provided to the bioreactor.

11. The method of claim 4 further comprising the production of hydrogen gas.

12. The method of claim 11 wherein the hydrogen gas and hydrogen dissolved in an aqueous solution is provided to the bioreactor directly from the electrolyzer stack.

13. An electrolyzer capable of producing pressurized hydrogen gas in an aqueous solution wherein the electrolyzer comprises a water/oxygen phase separator, a pump, an electrolyzer stack, and a back pressure regulator and wherein the electrolyzer does not comprise a hydrogen drying system or a hydrogen/water phase separator.

14. A system for the production of a gas comprising an electrolyzer capable of producing pressurized hydrogen gas in an aqueous solution wherein the electrolyzer comprises a water/oxygen phase separator, a pump, an electrolyzer stack, and a back pressure regulator and wherein the electrolyzer does not comprise a hydrogen drying system or a hydrogen/water phase separator and wherein the system further comprises a bioreactor that uses the pressurized hydrogen gas in an aqueous solution and a carbon containing gas and a biocatalyst in an aqueous solution to produce the gas.

15. The system of claim 14 wherein the gas is methane.

16. The system of claim 15 wherein the carbon containing gas is carbon dioxide.

17. The system of claim 15 wherein the biocatalyst is Methanothermobacter thermautotrophicus.

18. The system of claim 14 wherein the pressurized hydrogen gas in an aqueous solution is used to control the pH of an aqueous solution within the bioreactor.

19. The system of claim 14 wherein the pressurized hydrogen gas in an aqueous solution is alkaline.

20. The system of claim 19 wherein the aqueous solution comprises KOH or NaOH.
